(19) United States
(12) Patent Application Publication
(10) Pub. No.: US 2021/0350876 A1
(43) Pub. Date: Nov. 11, 2021

(54) SYSTEM AND METHOD FOR DIRECT SUBSEQUENCE SEARCHING AND MAPPING IN NANOPORE RAW SIGNAL

(71) Applicant: KING ABDULLAH UNIVERSITY OF SCIENCE AND TECHNOLOGY, Thuwal (SA)

(72) Inventors: Xin GAO, Thuwal (SA); Sheng WANG, Thuwal (SA); Renmin HAN, Thuwal (SA)

(21) Appl. No.: 17/285,255

(22) PCT Filed: Oct. 16, 2019
(86) PCT No.: PCT/IB2019/058829

§ 371 Date: Apr. 14, 2021

Related U.S. Application Data
(60) Provisional application No. 62/750,304, filed on Oct. 25, 2018.

(51) Int. Cl. G16B 40/10; G16B 30/10; G06K 9/62; G01N 33/487

(52) U.S. Cl. CPC G16B 40/10; G16B 30/10; G16B 40/20; G01N 33/48721; G06K 9/6215

(57) ABSTRACT

A method for similarity searching directly on nanopore raw current signals, the method including receiving a reference genome sequence; receiving a query genome sequence; transforming the reference genome sequence, with a nanopore sequencing device, into a raw current signal X; transforming the query genome sequence, based on a pore model, into a query current signal Y; and mapping the query current signal Y to the raw current signal X based on a subsequence extension of dynamic time warping distance Dist, which calculates a distance between the raw current signal X and a padded signal query Y′. The padded signal query Y′ is the query current signal Y to which an element y₀ has been added, the raw current signal X and the query current signal Y are electrical currents, and the raw current signal X corresponds to a genome of an organism.

### CROSS-REFERENCE TO RELATED APPLICATIONS

This application claims priority to U.S. Provisional Patent Application No. 62/750,304, filed on Oct. 25, 2018, entitled “TWO NOVEL SUBSEQUENCE DYNAMIC TIME WARPING ALGORITHMS FOR NANOPORE RAW SIGNAL SEARCH,” the disclosure of which is incorporated herein by reference in its entirety.

### BACKGROUND

### Technical Field

Embodiments of the subject matter disclosed herein generally relate to a system and method for nanopore raw signal search, and more particularly, to novel algorithms that enable direct subsequence inquiry and exact mapping in nanopore raw signal datasets.

### Discussion of the Background

Nanopore sequencing is a rapidly developing technology that is able to generate 10-50 k ultra-long reads in real time on a portable instrument at low-cost. The idea of the nanopore sequencing resides in pore chemistry, which directly measures the changes in the electrical current signal (called herein as the raw current signal) when a single-strand DNA (of any length) passes through a physical nanopore [1]. Long reads are extremely valuable to provide long-distance information on genome or transcriptome, which simplifies the genome/transcriptome assembly and the detection of large structural variance (SV).

However, it is challenging to use nanopore sequencing with high-coverage for the detection of single nucleotide polymorphisms (SNPs), which is the key for gene-disease diagnoses, association studies, ancestry inference, drug design, and linked region detection. One reason for this problem is due to the higher error rate (more than 10%) in the reads generated by nanopore sequencing compared to 1% error rate for 2nd generation sequencing technique that generates short seeds.

According to [2], there are two sources of errors in nanopore sequencing: (i) the inherent error that comes from the low signal-to-noise ratio in the raw current signal during the sequencing, and (ii) the base-calling error that comes from the process of translating the raw current signal into a DNA sequence by machine learning models, which depends heavily on the datasets that are used to train the parameters of the model. Note that base-calling is the computational process of translating raw current signals into a nucleotide sequence. Experience has shown that biases in the training data (such as the type of species) could result in errors when applying the machine learning model to base-call new data.

In the cases where a mutation or modification occurred in the nucleotide acids, the base-calling will lose non-standard information and hamper the further detection. Therefore, to keep the information underlying the raw current signal is very important. For example, [3] utilized the raw current signal to detect the DNA methylation via a hidden Markov model (HMM) model, and [4] proposed a method that may able to de novo detect the DNA modifications without any model training. The key underlying for these methods is the end-to-end mapping between the raw signal sequence and the reference genomic sequence, in which the nanopore raw signal is first translated to a read (i.e., nucleotide sequence) and then aligned with the reference genome.

However, such an approach still inevitably introduces base-calling errors and the information loss is irreversible. On the contrary, a reversible choice has been proposed by firstly translating the reference sequence to an expected signal via the official k-mer pore model and then operating between the two signals [5].

In most conditions, the biologists may have the idea of where the SNPs take place along the DNA strand. SNPs are the most common type of genetic variation among people. Each SNP represents a difference in a single DNA building block, the nucleotide. For example, a SNP may replace the nucleotide cytosine (C) with the nucleotide thymine (T) in a certain stretch of DNA. The biologists need a quick way to obtain the evidence about the locations of the SNPs along the DNA. With the help of nanopore sequencing, the problem of the detection of any SNP could be formulated as a signal search problem, or, more generally, as a subsequence inquiry.

Sequence similarity search and alignment is the fundamental problem in bioinformatics [6]. Classically, a DNA sequence is presented as a string of nucleic acids, which are labeled as {A, C, G, T}. The similarity search is carried out between two actual DNA sequences with certain labeled nucleic acids. Dynamic programming can be applied to produce global alignments via the Needleman-Wunsch algorithm, and the Smith-Waterman algorithm. However, there are many sequences in a dataset. To achieve a quick search, a variety of specialized methods have been proposed. Most of the methods are implemented based on k-mer hashing, among which BLAST and BLAT are the most popular similarity search tools. DALIGNER uses a sort and merge paradigm to fast determine the local alignment. MHAP and Mashmap adopt the MinHash-based estimation. Minmap is the joint of these methods with very fast speed.

Compared with the above well-developed genome sequence alignment methods, there is little development on the similarity search of signal presented genome. However, the signal and time-series similarity search also has been studied for decades. Unlike the DNA sequence, which has exact and discrete values for each element, the signal sequence contains numerous elements with a wide range of values. Given a query sequence, the aim of the similarity search method in a signal and time-series is to find all the sequences or subsequences that are close to the query sequence within a given distance.

The most widely used distance measure is the Euclidean distance. However, the Euclidean distance is not supported by those sequences that have insertion and deletion. Dynamic Time Warping (DTW) is a more robust approach that allows time-axis scaling [7]. Though DTW has been well-established, the original DTW has a O(n²) time complexity, which means that it requires intensive computational power. To address this cost issue, several methods have been proposed to constrain the calculations, such as boundary-constrain [8], coarsening [9], and multi-scale [10], [11]. The subsequence matching based on DTW has been widely used in similarity search over data streams [13], [14], [15].

However, the above methods are still computer intensive, slow, and/or unreliable. Thus, there is a need for a method that overcomes the above noted disadvantages.

### BRIEF SUMMARY OF THE INVENTION

According to an embodiment, there is a method for similarity searching directly on nanopore raw current signals. The method includes receiving a reference genome sequence, receiving a query genome sequence, transforming the reference genome sequence, with a nanopore sequencing device, into a raw current signal X, transforming the query genome sequence, based on a pore model, into a query current signal Y, and mapping the query current signal Y to the raw current signal X based on a subsequence extension of dynamic time warping distance Dist, which calculates a distance between the raw current signal X and a padded signal query Y′. The padded signal query Y′ is the query current signal Y to which an element y₀ has been added, the raw current signal X and the query current signal Y are electrical currents, and the raw current signal X corresponds to a genome of an organism.

According to another embodiment, there is a computing device for similarity searching directly on nanopore raw current signals. The device includes an input/output interface configured to receive a reference genome sequence, and a query genome sequence, and a processor connected to the input/output interface. The processor is configured to transform the reference genome sequence, with a nanopore sequencing device, into a raw current signal X, transform the query genome sequence, based on a pore model, into a query current signal Y, and map the query current signal Y to the raw current signal X based on a direct subsequence dynamic time warping distance Dist, which is configured to calculate a distance between the raw current signal X and a padded signal query Y′. The padded signal query Y′ is the query current signal Y to which an element y₀ has been added, the raw current signal X and the query current signal Y are electrical currents, and the raw current signal X corresponds to a genome of an organism.

According to still another embodiment, there is a method for similarity searching directly on nanopore raw current signals, the method including receiving a raw current signal X, receiving a query current signal Y, extracting feature signals X′^{L} from the raw current signal X, and feature signals Y^{L} from the query current signal Y, calculating a coarse path W_{coarse} by applying a direct sequence dynamic time warping distance DSDTW to (i) the feature signals X′^{L} and Y^{L}, and (ii) to plural seeds Q, wherein the coarse path W_{coarse} is a list that maps elements of the raw current signal X to the query current signal Y, and applying a continuous wavelet-based multi-level transform cwDTW to the coarse path W_{coarse} to obtain a fine path W_{fine}. The fine path W_{fine} is a list that more accurately maps elements of the raw current signal X to the query current signal Y, then the coarse path W_{coarse}.

In yet another embodiment, there is a computing device for similarity searching directly on nanopore raw current signals, the computing device including an input/output interface configured to receive a raw current signal X and to receive a query current signal Y, and a processor connected to the input/output interface. The processor is configured to extract feature signals X′^{L} from the raw current signal X, and feature signals Y^{L} from the query current signal Y, calculate a coarse path W_{coarse} by applying a direct sequence dynamic time warping distance DSDTW to (i) the feature signals X′^{L} and Y^{L}, and (ii) to plural seeds Q, wherein the coarse path W_{coarse} is a list that maps elements of the raw current signal X to the query current signal Y, and apply a continuous wavelet-based multi-level transform cwDTW to the coarse path W_{coarse} to obtain a fine path Wane. The fine path W_{fine} is a list that more accurately maps elements of the raw current signal X to the query current signal Y, then the coarse path W_{coarse}.

### BRIEF DESCRIPTION OF THE DRAWINGS

For a more complete understanding of the present invention, reference is now made to the following descriptions taken in conjunction with the accompanying drawings, in which:

FIG. 1 illustrates a reference genome sequence, a query sequence, and associated raw current signals;

FIG. 2 illustrates aligned raw current signals for two queries;

FIG. 3A illustrates an Euclidian distance calculated for a query and a resampled raw current signal, and FIG. 3B illustrates a dynamic time warping distance calculated for the query and the original raw current signal when selected with a slide window;

FIG. 4A illustrates a subsequence-extended dynamic time warping distance calculated for the query and the original raw current signal, and FIG. 4B illustrates the subsequence-extended dynamic time warping distance calculated for the query and the resampled raw current signal;

FIG. 5 schematically illustrates an algorithm that implements the subsequence-extended dynamic time warping distance;

FIG. 6 illustrates the influence of the query length on the subsequence-extended dynamic time warping distance;

FIG. 7 illustrates the relationship between the mapped path of three seeds extracted from a long query;

FIG. 8 is a flow chart of a method for calculating a correlation coefficient between the seeds and the linear constraint;

FIG. 9 illustrates an algorithm that implements the continuous wavelet-based multi-level dynamic time warping distance;

FIG. 10 is a flow chart of a method that uses a subsequence-extended dynamic time warping distance for mapping a query current signal to a raw current signal;

FIG. 11 is a flow chart of a method that uses a continuous wavelet-based multi-level dynamic time warping distance for mapping a query current signal to a raw current signal;

FIG. 12A illustrates the query current signal and the raw current signal and FIG. 12B illustrates the detailed current changes corresponding to the query current signal and the raw current signal, when the continuous wavelet-based multi-level dynamic time warping distance is used;

FIG. 13A illustrates the distribution of the edit mapping error for two novel methods and FIG. 13B is a scatter plot between the edit mapping error for the two methods;

FIG. 14A illustrates the average edit mapping error of the query for the Human 21 database and FIG. 14B illustrates the average edit mapping error of the query for the Lambda phase database when the two novel methods are applied;

FIG. 15 illustrates the average edit mapping error of the query results for different parameters of the used method;

FIG. 16A illustrates the effect of the query length on the runtime of the two methods and FIG. 16B illustrates the effect of the raw current signal length on the runtime of the two methods; and

FIG. 17 is a schematic diagram of a computing system that is used to implement one or more of the methods discussed herein.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel algorithm/method, the Direct Subsequence Dynamic Time Warping for nanopore raw signal search (DSDTWnano) enables direct subsequence inquiry and exact mapping in the nanopore raw signal datasets. The proposed algorithm is based on the idea of Subsequence-extended Dynamic Time Warping (SDTW) and directly operates on the raw current signals, without any loss of information. The DSDTWnano method could ensure highly-accurate query results.

According to another embodiment, another novel algorithm/method, the continuous wavelet Subsequence DTW for nanopore raw signal search (cwSDTWnano) is introduced and enables the direct subsequence inquiry and exact mapping in the nanopore raw current signal datasets. The cwSDTWnano method is the accelerated version of the DSDTWnano method, and this advantage is obtained with the help of seeding and multi-scale coarsening of signals based on continuous wavelet transform (CWT).

Furthermore, according to another embodiment, a novel error measurement is proposed to specify the mapping accuracy between a genome sequence and a raw current signal, which may serve as the standard criteria for further genome-to-signal mapping analysis.

Comprehensive experiments on three real-world nanopore datasets (Human, Lambda phage, and E. coli) have been performed with the novel algorithms and the results of these experiments demonstrate the efficiency and effectiveness of the proposed algorithms. In particular, a successful SNP detection is demonstrated, based on the cwSDTWnano algorithm on a relatively low coverage (20×), with a detection rate better than 95%.

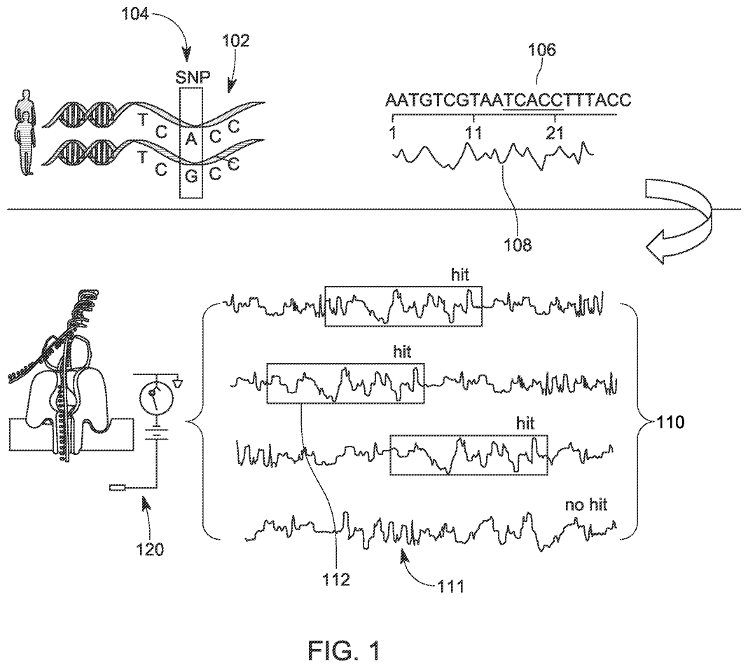

The inputs for these novel algorithms are (i) a candidate reference genome sequence 102 (where the genome sequence is associated with an organism, unicellular or multi-cellular), as illustrated in FIG. 1, in which either an SNPs 104 or another event exists, and (ii) a database 110 of nanopore raw current signals 111 that have corresponding regions 112 (from the view of DNA sequence) that correspond to the reference sequence 102. Note that FIG. 1 also shows a nanopore sequencing device 120 (for example, MinION) that uses as input the DNA genome and generates the raw current signals 111 (electrical current signals that correspond to the nucleotides of the DNS sequence). From the reference DNA sequence 102, a query sequence 106 is determined and, using a k-mer pore model (see, for example, https://github.com/nanoporetech/kmer_models), it is possible obtain a raw electrical current signal 108 (called query signal herein) that corresponds to the query sequence 106. Note that the query sequence 106 has only four possible values, i.e., the values T, C, A, and G of the four existing nucleotides, while the corresponding query signal 108 includes an infinite number of values corresponding to various electrical currents.

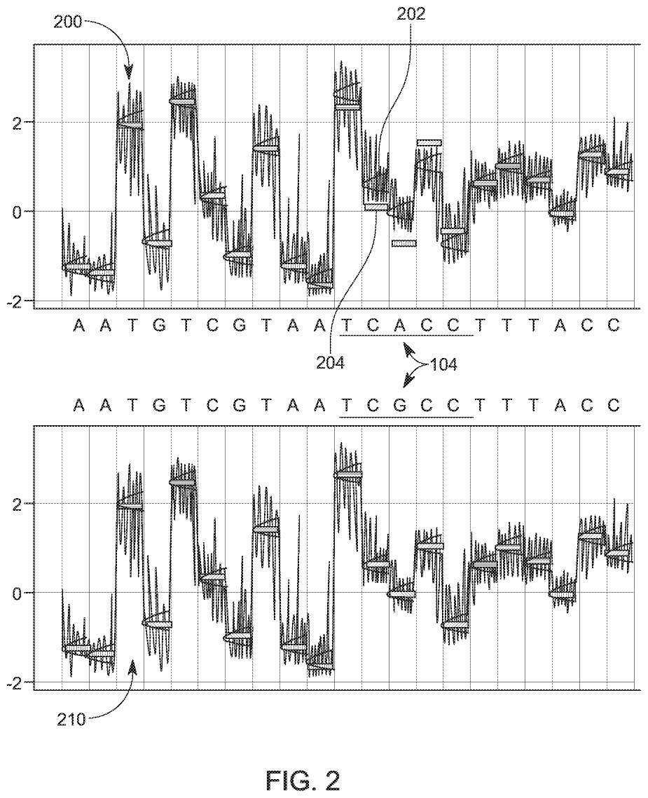

FIG. 2 illustrates the aligned raw signals 200 and 210 for two sequences with a SNP (here is A→G). A difference between the measured current signal 202 and the expected current signal 204, which is inferred from the pore model, indicates a strong evidence of the SNP 104.

The aim of this approach is to detect the segments 112 of the raw signals in the database 110, and map them onto the reference sequence 102 to discover evidence about the location of the SNPs 104. Most of the traditional methods make use of the base-called reads (genome sequence), which may have lost the special information underlying the raw current signal [2]. However, though there are various methods for similarity search and mapping on the genome sequences [16], [17], [18], there are no applicable methods for the direct similarity inquiry of the nanopore raw current signal 111.

The first method DSDTWnano is based on an effective subsequence-extended dynamic time warping distance and the boundary constrained dynamic time warping to ensure the accuracy mapping between the query signal and nanopore raw current signal. A mathematical foundation for this method is now discussed.

Let X=(x₁, x₂, . . . x_{N}) be the raw current signal 111 and Y=(y₁, y₂, . . . y_{M}) be the query signal 108, where M<N and the sampling rates of the X signal is higher (e.g., 8 or 9 times higher) than the sampling rates of the Y signal. The aim of the DSDTWnano method is to find a subsequence X[tₛ: tₑ]=(xₜ_{ₛ}, . . . , xₜ_{ₑ}) of X, with 1≤iₛ<tₑ≤N, where iₛ is a start time and tₑ is an end time, that minimizes a distance Dist measured between the query signal Y and each possible subsequence of X, i.e., a cost function E is minimized for a distance between Y and X according to equation (1):

$$E ( {t}_{s}^{opt} , {t}_{e}^{opt} ) = {arg min}_{( {t}_{s} {t}_{e} ) \text{:} 1 \leq {t}_{s} \leq {t}_{e} \leq N} Dist ( Y , X [ {t}_{s} \text{:} {t}_{e} ] ) . \qquad ( 1 )$$

As previously discussed, using an Euclidian measure (distance) for comparing two different sequences is not feasible for DNA sequences because such sequences can have additional or deleted elements. Thus, a dynamic time warping (DTW) is one of the algorithms used in time-series analysis for measuring a similarity (distance) between two temporal sequences that vary in speed. Given the two sequences X=(x₁, x₂, . . . x_{N}) and Y=(y₁, y₂, . . . Y_{M}), the DTW distance Dist(X,Y) between these two sequences is iteratively defined as:

$$\{ \begin{matrix}Dist ( X , Y ) = D ( N , M ) ; \\ D ( i , j ) = ‖ {x}_{i} - {y}_{j} ‖ \\ + min \{ D ( i - 1 , j ) , D ( i , j - 1 ) , D ( i - 1 , j - 1 \} ; \\ D ( 0 , 0 ) = 0 ; \\ D ( i , 0 ) = D ( 0 , j ) = ∞\end{matrix} \qquad ( 2 )$$

In one application, the DTW distance Dist(X,Y) can be exactly solved by dynamic programming, resulting in the globally optimal mapping. The DTW process requires O(NM) time for the calculation of the distance matrix with the N×M dimension (the space complexity could be O(M) if the warp path is not necessary to be backtracked). For the subsequence similarity search, a naive solution is to open a slide window for each location in the long sequence and calculate the DTW distance for each slide window, which would result in O(N²M) complexity.

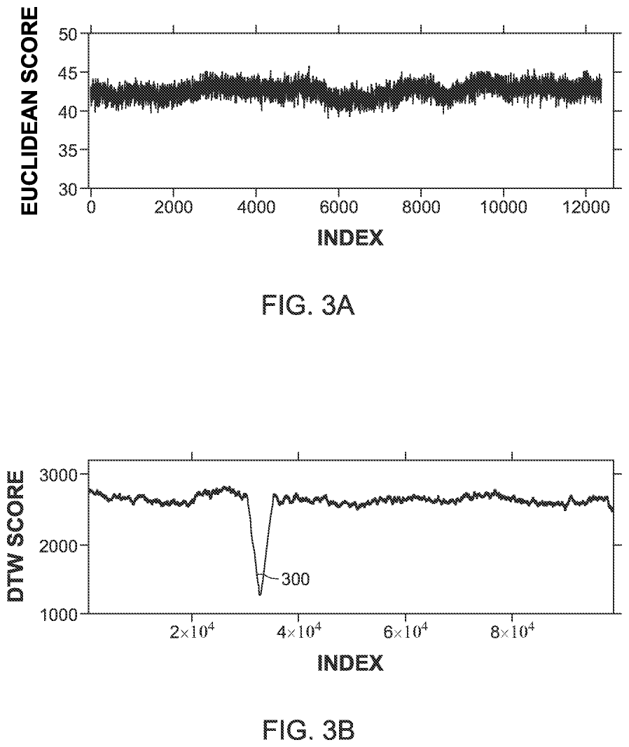

However, the inventors have observed that the DTW distance is able to return a distinguished distance for the regions of high similarity in the nanopore raw signal, which the Euclidean approach cannot. In this regard, FIG. 3A shows a similarity search performed using the Euclidean distance between the query Y and a resampled raw signal X′, which was selected from a slide window, in which the length of the slide window is the same as the length of the query, and FIG. 3B illustrates a similarity search performed using the direct DTW distance between the query Y and the raw signal X, which was selected from a slide window, in which the length of slide window is defined as 8× as the one of the query. Note that FIG. 3B shows a clear peak 300, which is an indication of a similarity between the query Y and the raw signal X while FIG. 3A show no peaks. However, the high-complexity of the DTW distance hampers its further application to practical applications.

A subsequence extension of the DTW distance is now discussed. A change could be applied to the primary DTW distance for the purpose of subsequence searching [15], if an omission in the alignment between X and Y, which was caused by the element repeating of X, is not considered. More specifically, given a query sequence Y=(y₁, y₂, . . . y_{M}) and a reference sequence X=(x₁, x₂, . . . x_{N}), it is possible to pad the query Y to obtain a padded query Y′=(y₀, y₁, y₂, . . . y_{M}), and define the added element y₀ as respecting the following condition ∥y₀,xᵢ∥=0 for all xᵢ. Consequently, the minimum distance Dist(X[tₛ: tₑ], Y) between the reference sequence X and the padded query Y′ could be derived from equations (2) as follow:

$$\{ \begin{matrix}Dist ( X [ {t}_{s} \text{:} {t}_{e} ] , {Y}^{′} ) = D ( {t}_{e} , M ) = min ( D ( t , M ) ) ; \\ D ( t , j ) = ‖ {x}_{t} - {y}_{j} ‖ \\ + min \{ D ( t - 1 , j ) , D ( t , j - 1 ) , D ( t - 1 , j - 1 ) \} ; \\ D ( t , 0 ) = 0 , D ( 0 , 1 ) = ∞ .\end{matrix} \qquad ( 3 )$$

After the calculation of the entire distance matrix given by equation (3), it is necessary to search along the D(t, M) path to find the end point to and then, to track back to the start point tₛ. The total complexity of the subsequence extension DTW remains as O(NM). However, it should be noted that the extended version of the DTW distance is sensitive to the omission of the element repeating along the time axis of sequence X.

Because a similarity search directly on nanopore raw signals has never been performed before, an experiment was designed to investigate the effectiveness of different distance measurements and different implementations of the DTW distance when applied between the query signal Y and raw current signal X (as previously discussed, the query signal Y is obtained from the query genome based on the official k-mer pore model). It is noted that the nanopore raw signal has a much higher (e.g., 8 to 9 times higher) redundancy of the sampling ratio relative to the query signal. To make this comparison more comprehensive, the similarity was calculated directly on the raw current signal X as well as on the resampled signal X′. A 8-times compressed raw signal X′ (resampled signal) is generated from the raw current signal X by using a FIR-based (finite-impulse response filter) resampling technique [19]. A query signal Y with 1,000 nucleic acids and a nanopore raw current signal X with ˜100,000 elements are selected to demonstrate the power of the subsequence extension DTW distance.

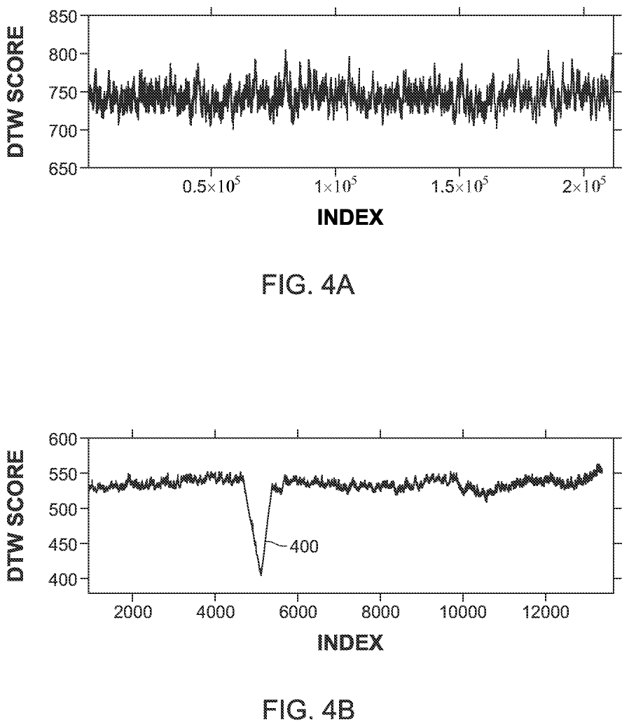

FIG. 4A illustrates a similarity search performed using the subsequence-extended DTW distance (also called score) between the query Y and the raw current signal X, i.e., the D(t, M) along the time axis while FIG. 4B illustrates the similarity search performed with the subsequence-extended DTW distance between the query Y and the resampled raw signal X′.

As previously discussed with regard to the traditional DTW distance, in FIG. 3A, the Euclidean distance has no effect even when the nanopore raw current signal 111 has been compressed to a similar scale as the query signal 108. On the contrary, FIG. 3B shows that the DTW distance could give out a distinguishable response (peak 300) for the high similarity regions. However, the slide window based search strategy used for the results shown in FIGS. 3A and 3B costs half an hour to get the result and is quite unacceptable for practical applications. However, in FIG. 4A, the subsequence-extended DTW similarity has been used to accelerate the search, but the zero padding has no effect on the nanopore raw signal, and thus it results in a bad measurement. The reason why the subsequence-extended DTW similarity failed for the nanopore raw current signal 111 in FIG. 4A is that the original nanopore raw signal has massive repeatings (redundant sampling), which makes the data to disobey the assumption that there are no omissions in the alignment between the query sequence 106 and the reference sequence 102. Consequently, the subsequence-extended DTW similarity was run between the query Y and the resampled raw signal X′, which resulted in a sharp peak 400, as shown in FIG. 4B. With the introduction of the subsequence-extended DTW distance, the complexity of calculating the similarity has been reduced from O(N²M) to O(NM), and the runtime is reduced from hours to seconds (e.g., 1˜2 seconds).

Based on the observations of this experiment, it can be concluded that a subsequence with high-similarity to a query could be discovered by a peak picking algorithm on a resampled (compressed) nanopore raw signal, as illustrated in FIG. 4B. However, the difficulty of applying the subsequence-extended DTW distance on the nanopore raw signal data 110 is the scale difference between the query signal 108 and the raw current signal 111. For this case, it is possible to first use a resampled nanopore raw current signal X′, and then extend it to a finer case. An early determination strategy can be introduced into the procedure to protect from inefficient computation. For example, the optimal location could be judged as a peak if D (t, M)<ϵ, where the threshold E could be determined from the historic record of D (t, i) or the statistics of the previous experiments. Consequently, the DSDTWnano algorithm is designed to cope with such a short seeds inquiry.

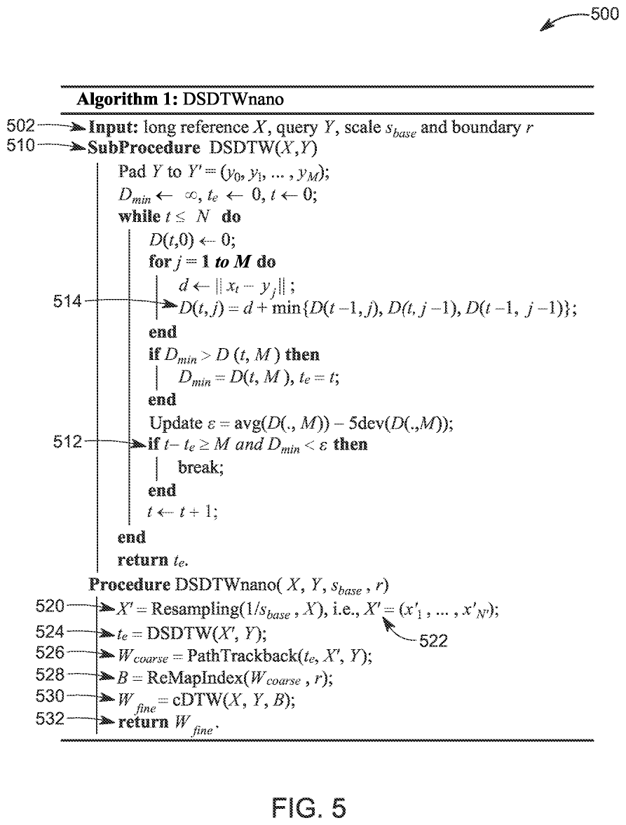

The DSDTWnano algorithm is now discussed in more detail with regard to FIG. 5, which shows a computer software implementation. The DSDTWnano algorithm 500 relies on a subprocedure DSDTW(⋅) 510, which is the subsequence-extended dynamic time warping distance with an early stop condition 512. The subprocedure 510 reports the end location tₑ of the high-similarity region between a raw current signal X and a padded query signal Y′. The distance 514 used by this subprocedure is the one defined by equation (3). The threshold E is defined in this implementation as being the difference between (i) the average of the distance and (ii) the five times the number of deviations of the distance.

The DSDTWnano algorithm 500 starts in step 502 by receiving the raw current signal X, the query signal Y, a scale s_{base} (which is related to the resampled raw current signal X′), and a boundary r. Then, in step 520, the raw signal X 111, which is obtained as discussed above with regard to FIG. 1, is resampled using the function Resampling(⋅) which is the FIR-based resampling to compress the nanopore raw signal X, as disclosed, for example, in [19]. Other procedures/algorithms may be used for resampling the raw current signal X. Note that the resampling function generates the resampled raw current signal X′ 522 based on the raw signal X 111 and the scale s_{base}. In step 524, the end point tₑ of the coincident part of the resampled raw signal X′ and the query signal Y is determined using the subprocedure 510 discussed above. Then, in step 526, a coarse path (or mapping) W_{coarse} (i.e., a list) between the resampled raw signal X′ and the query signal Y is found, starting from the end point tₑ, using for example, the function PathTrackback(⋅) This is a function that recursively search the match paths between X′ and Y that starts from tₑ. Having the end point tₑ, the method makes continuous the coarse path W_{coarse} in step 528, using a function ReMapindex(⋅) which is a context-dependent constraint generation function that generates a list or map B from the coarse path W_{coarse}, with a window size r. In step 530, the method calculates a fine path W_{fine} between the X and Y signals, using the subprocedure cDTW(⋅), which is the constrained dynamic time warping (see, for example, [20]). The method returns in step 532 the fine path W_{fine}, which is a list or mapping between the query Y and the raw signal X.

Because the subsequence-extended DTW distance has the complexity of O(MN′) and the constrained DTW distance has the complexity of O(rM), the overall complexity of the DSDTWnano method is

$$0 ( \frac{1}{{s}_{base}} MN ) .$$

Although the DSDTWnano method 500 is better when compared to the slide-window based similarity search, it is still slow when the query signal or reference sequence has a quite long length. Thus, several techniques are implemented to further speedup the similarity search on the nanopore raw signal, such as seeding, pre-filtering, and multi-scale search.

The high-noise and time-axis warping of the nanopore raw signal make it impossible to design a hash-table for a “k-mer” search. However, the inventors have observed the existence of a minimum length with a high correctness for a sensitive similarity search.

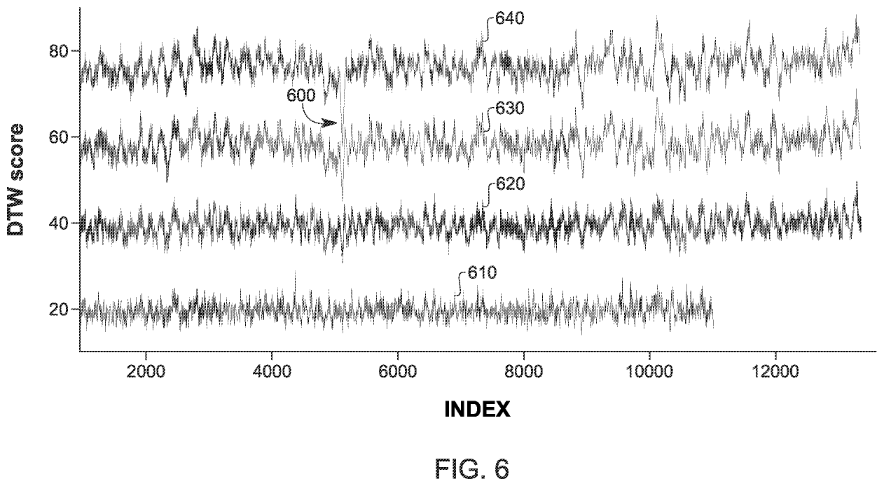

In this regard, the inventors have performed the following calculations to determine how the length of the query signal Y affects the similarity detection in the DSDTWnano method. This investigation focused on the compressed nanopore raw signal X′ and a short query signal Y with 32, 64, 96 and 128-long (all the reads have the same suffix). FIG. 6, which plots the DTW score (or distance) versus the index of the reads, shows some of the results of this investigation. Note that curve 610 corresponds to a 32-mer, curve 620 corresponds to a 64-mer, curve 630 corresponds to a 96-mer, and curve 640 corresponds to a 128-mer. It can be seen that the 32 or 64-long queries 610 and 620 cannot generate a distinguishable response with the compressed raw signal X′. This result has two reasons: (i) the noise residing in the raw current signal degenerated the true response, and (ii) the existence of multiple similar segmentation on the raw signal. However, when the query length grows, for example, to the 96 and 128-long queries 630 and 640 in FIG. 6, the true response 600 emerges.

Based on this investigation, the inventors have determined that a read with a length equal to or larger than 128 is generally long enough to be detected on the nanopore raw current signal X with a high confidence. The inventors have tested a number of reads and proved that a length of 128 is enough to obtain a confident response based on the subsequence direct DTW distance. Herein, such a short length is called the minimal length.

Thus, it is postulated that a minimal length always exists for a set of nanopore raw signals 111 that are collected using the same configuration. To justify this assertion, the following derivation is presented. From the k-mer model provided by the Nanopore technology, a measured current signal X belongs to one of the random variables {S₁, S₂, . . . , S_{N}}, in which Sᵢ˜ [symbol] (μᵢ, σᵢ²) for each Sᵢ (N=4⁵ for the 5-mer model and 4⁶ for the 6-mer model). For the convenience of the discussion, it is assumed that the variance of different Sᵢ has a similar value, i.e., ∀i, σ≈σᵢ. If x is a measured value of Sᵢ and y is a measured value of Sⱼ, the statistic of x−j will belong to Dᵢⱼ=Sᵢ—Sⱼ and Dᵢⱼ˜ [symbol] (μᵢ−μⱼ, σᵢ²+σⱼ²), according to the property of Gaussian distribution. Specifically, it is found that Dᵢⱼ˜ [symbol] (0,σᵢ²+σⱼ²) if i=j.

Next, consider the distance of the DTW path { [symbol] i₁, j₁ [symbol] [symbol] i₂, j₂ [symbol] , . . . [symbol] i_{K}, j_{K} [symbol] }, where K is the length of the path. Because an omission is not considered in the subsequence-extended DTW distance, it is assumed that the length of the detected paths for different locations are the same. Thus, the subsequence-extended DTW distance becomes:

$$Dist ( X , Y ) = {\sum }_{k = 1}^{K} \vert {x}_{{i}_{k}} - {y}_{{i}_{k}} \vert . \qquad ( 4 )$$

The distance Dist(X, Y) behaves as a random variable in statistics, which can be described by:

$$C = {\sum }_{i = 1}^{N} {\sum }_{j = 1}^{N} ( {α}_{ij} \vert {D}_{ij} ) , \qquad ( 5 )$$

where Σᵢ₌₁^{N} Σⱼ₌₁^{N}αᵢⱼ=K and αᵢⱼ is a positive integer value which indicates how many times Dᵢⱼ appeared in the path. Because Dᵢⱼ follows a Gaussian distribution, the random variables Dᵢⱼ will have a normal distribution. For the convenience of the discussion, the following notations are introduced: μᵢⱼ=μᵢ−μⱼ and ω=σᵢ²+σⱼ². Then,

$${μ}_{\vert {D}_{ij} \vert} = ω \sqrt{\frac{2}{π}} exp ( \frac{- {μ}_{ij}^{2}}{2 {ω}^{2}} ) + {μ}_{ij} erf ( \frac{{μ}_{ij}}{\sqrt{2 {ω}^{2}}} ) \qquad ( 6 )$$

and

σ²_{|D}_{ᵢⱼ}_{|}μᵢⱼ²+ω²−μ²_{|D}_{ᵢⱼ}_{|} (7)

where

$$erf ( z ) = \sqrt{\frac{2}{π}} {\int }_{0}^{z} {e}^{- {t}^{2}} d t$$

is the error function. Based on the properties of the extended-sequence DTW distance, the following relationships are true:

$${μ}_{\vert {D}_{i i} \vert} = ω \sqrt{\frac{2}{π}} and {σ}_{\vert {D}_{ij} \vert}^{2} = ( 1 - \frac{2}{π} ) {ω}^{2}$$

as μᵢᵢ=0 for i=j.

The derivative of μ_{|d}_{ᵢⱼ}_{|} is given by:

$${μ}_{\vert {D}_{ij} \vert}^{′} ( {μ}_{ij} ) = ω \sqrt{\frac{2}{π}} \frac{d}{d {μ}_{i j}} exp ( \frac{- {μ}_{i j}^{2}}{2 {ω}^{2}} ) + erf ( \frac{{μ}_{i j}}{\sqrt{2 {ω}^{2}}} ) \frac{d}{d {μ}_{i j}} {μ}_{i j} + {μ}_{i j} \frac{d}{d {μ}_{i j}} erf ( \frac{{μ}_{i j}}{\sqrt{2 {ω}^{2}}} ) = erf ( \frac{{μ}_{i j}}{\sqrt{2 {ω}^{2}}} ) . \qquad ( 8 )$$

Based on this relationship, it is noted that μ_{|D}^{ij}| decreases when μᵢⱼ<0 and increases when μᵢⱼ>0 as erf is an odd function and larger or equal to zero on the right axis, which means that μ_{|D}_{ᵢⱼ}_{|} gets its minimum value at μᵢⱼ=0. Therefore, μ_{|D}_{ᵢᵢ}_{|}≤μ_{|D}_{ᵢⱼ}_{| for all i and j.}

Returning to the DTW path, the following quantities are introduced,

$${\overline{μ}}_{\vert {D}_{ij} \vert} = \frac{1}{{N}^{2}} {\sum }_{i = 1}^{N} {\sum }_{j = 1}^{N} ( μ \vert {D}_{i j} ) and {\overline{σ}}_{\vert {D}_{ij} \vert} = \frac{1}{{N}^{2}} {\sum }_{i = 1}^{N} {\sum }_{j = 1}^{N} ( σ \vert {D}_{i j} )$$

and it is assumed that the elements that are mismatched along the path are fully random (random variable C_{mismatch}) and the elements that are well-matched along the path have a p sequence error given by 0≤p≤1 (i.e., a random variable C_{match}). By relaxing the computation of E(C), the following equation is obtained:

E(C_{mismatch})=Kμ̅_{|D}_{ᵢⱼ}_{|} (9)

and

E(C_{match})=p·Kμ̅_{|D}_{ᵢⱼ}_{|}+(1−p)Kμ_{|D}_{ᵢⱼ}_{|} (10)

Then, by combining equations (9) and (10), the following equation is obtained: E(C_{mismatch})−E(C_{match})=(1−p)K(μ̅_{|D}_{ᵢⱼ}_{|}−μ_{|D}_{ᵢⱼ}_{|}). Thus, a minimal length value of K for sensitive similarity is given by:

$$K = \frac{L}{( 1 - p ) ( {\overline{μ}}_{\vert {D}_{ij} \vert} - {μ}_{\vert {D}_{ij} \vert} )} \qquad ( 11 )$$

and this value ensures that a short read is detected with high confidence (where L is a sensitive value). When implementing the minimum length defined by equation (11), the DSTWnano method becomes faster and more accurate.

Another improvement to the DSDTWnano method may be implemented with regard to the filtering of the false alignment with minimal length. In this regard, for a query signal that has a length of more than 1,000, it is feasible to use seeds with the minimal length K to accelerate the query speed. In other words, given a query signal Y, several segments Q of the query signal Y, each having the minimal length K previously discussed, could be selected. Two conditions are imposed onto these segments Qᵢ: (i) if the query signal Y has a high similarity region with the raw current signal X, the segments Qᵢ with the minimal length could also have a distinguishable response with the raw current signal X, and (ii) if the query signal Y does not have a high similarity region with the raw current signal X, a picked response with the minimal length will contain no useful information.

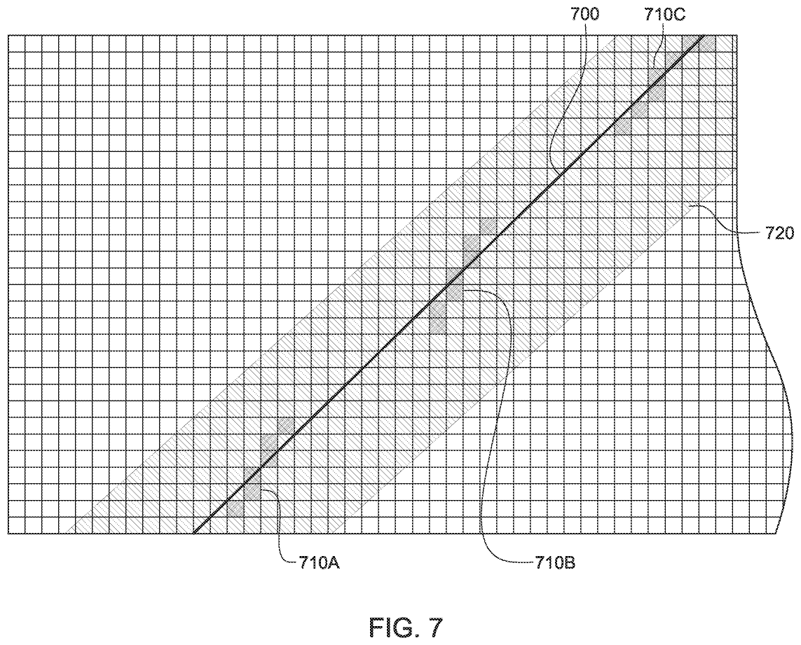

FIG. 7 shows the mapping between the nanopore raw current signal X (line 700) and three short seeds 710A to 710C with the minimal length K, which were extracted from a relative long query Y, in which the mapping paths are constrained within a linear boundary 720. Based on the above observations, the following filtering operation is developed to quickly exclude the portions of the raw current signal that have no high-similarity responses with the query signal:

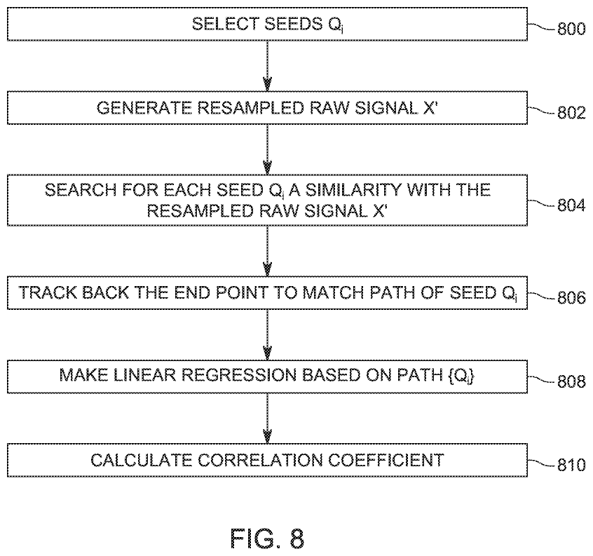

In step 800, as illustrated in FIG. 8, the method selects a set of segments {Qᵢ}_{i=1, . . . , j} from the query signal Y, to be the seeds. The upper number J may have any value (e.g., in the hundreds). In step 802, the method generates the resampled raw signal X′, as previously discussed. In step 804, for each seed Qᵢ, the method searches on the resampled (or compressed) raw signal X′ using the procedure DSDTW( ) discussed above with regard to FIG. 5, to obtain a location with a distinguishable response (i.e., a clear peak). Then, in step 806, the method tracks back the end point to obtain the match path for each seed Qᵢ. In step 808, a linear regression is applied to the paths of the set of seeds {Qᵢ} and in step 810 a correlation coefficient is calculated between the seeds and the linear constraint, so that the process can be stopped if the coefficient is too small.

With the filtering operation discussed with regard to FIG. 8, if the linear relationship of the seeds has been violated, the DSDTWnano method can be configured to be stopped to save computational time. If the selected J seeds each has a K length, the total cost for a N-long raw current signal is

$$O ( \frac{N}{{s}_{b a s e}} KJ ) ,$$

which is relative small as K and J are quite small compared to the length of a long query Y.

A second algorithm/method for searching a similarity directly on electrical current signals, between a raw current signal and a query signal, different from the DSDTWnano method discussed above, that can obtain a similar output but at a faster pace, is the continuous wavelet Subsequence DTW for nanopore raw signal search (cwSDTWnano). The cwSDTWnano method is an accelerated version of the DSDTWnano method. In essence, the cwSDTWnano method starts from a discrete seed search on coarsened raw signals to filter out the false candidates. Then, a low-resolution wavelet transforms is imposed on the ultra-long nanopore current signal and the subsequence query such that the data information is highly compressed. With a subsequence time warping, a set of very coarsened mapping patches is produced and combined with the picked seeds so that a fuzzy mapping path (W_{coarse}) can be established. Then, with a multi-scale coarsening and remapping technique, which is based on the continuous wavelet technique, the warping path W_{coarse} is recursively projected from a lower-resolution level to a higher-resolution to obtain a fine path W_{fine}. Details of this method are now discussed.

In this regard, when handling long-signal sequences, down-sampling combining with multi-scale analysis has been used to decrease the execution time. Further, the wavelets are usually used in the multi-scale analysis as well as preserve the feature information. However, the multi-scale analysis is hard to be implemented in the DTW-based subsequence search task because it cannot preserve the uniqueness of the high-similarity region.

In this embodiment, with the aid of the minimal length seeds and the most recent multi-scale schedule [21], the cwSDTWnano method is capable to further accelerate the similarity search for nanopore raw signal as now discussed.

A continuous wavelet transform (CWT) is used to divide a continuous-time function into wavelets. In particular, the CWT of a one-dimensional signal X(t) at a scale aϵ [symbol] ⁺ and translational value bϵ [symbol] , denoted as X_{a,b}, is expressed by the following integral:

$${X}_{a , b} = \frac{1}{\sqrt{a}} {\int }_{- ∞}^{∞} X ( t ) {ψ}_{a , b} ( t ) d t , \qquad ( 12 )$$

where

$${ψ}_{a , b} ( t ) = ψ ( \frac{t - b}{a} )$$

is the mother wavelet, which is a continuous function in both the time domain and the frequency domain. In the method discussed herein, the Mexican hat wavelet ψ(t)=(1−t²)exp^{-t}^{²}^{/2} is the default mother wavelet. However, one skilled in the art will understand that other wavelet functions may also be used instead of the Mexican hat wavelet.

Regarding the multi-level representation of the cwSDTWnano method, for the convenience of the analysis, the translational value b was fixed in this embodiment to be the same as the index of the corresponding raw current signal X. That is, the transformed signals (spectrum) have the same length and retain peer-to-peer index to the raw signal X. The CWT(X, a) denotes in this embodiment the transformed spectrum of the raw signal X with the scale parameter a.

According to the work of [5], CWT can preserve the feature information for nanopore raw current signal. A feature extraction procedure can be carried out (denoted as PickPeaks(⋅) to reduce the length of the raw signal X as follows: (i) obtain the spectra CWT(X, a) from the raw current signal X; (ii) normalize the spectra CWT(X, a) based on Z-score normalization; and (iii) extract peaks and nadirs from each spectrum as the feature sequence. In this way, the length of a signal X could be reduced more than a times for a classic nanopore raw current signal X.

A coarse path may be determined using the CWT and the seeds previously discussed. In this regard, a set of minimal length seeds {Qᵢ} is used and their corresponding paths with the resampled raw signal X′ are recorded. These short paths can be used as anchors in the consequent mapping, as discussed later.

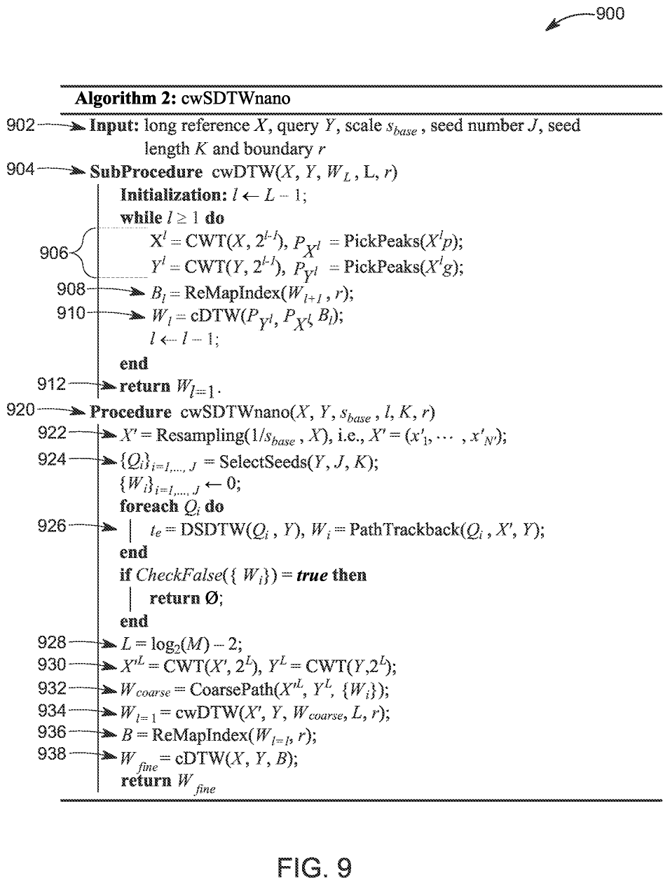

However, there are several long gaps between these short paths. Thus, the coarse paths generated by a continuous wavelet based subsequence DTW is used to fill these gaps (query signal Y and compressed raw signal X′). A method 900 that achieves these results is now discussed with regard to FIG. 9. In step 902, the long reference raw current signal X and the query signal Y are provided as input. Note that both signals X and Y are electrical signals measured with the nanopore sequencing device 120 shown in FIG. 1. In addition, in this step, the scale s_{base}, the seed number J, the seed length K, and the boundary r may also be input. A continuous wavelet DTW sub-procedure, called herein cwDTW, is defined in step 904. This sub-procedure extracts in step 906 feature information, using functions PickPeaks, from the continuous wavelet transform of the signals X and Y. In step 908, the procedure calculates a path Bi, similar to step 528 discussed above with regard to FIG. 5, based on the extracted features. The path Bi is then further refined in step 910, by applying the constrained DTW function cDTW, also discussed in FIG. 5, step 530, to obtain a finer path W_{I}, which is used later in the method. Note that in one application, the cDTW function is the continuous wavelet-based multi-level dynamic time warping discussed in [5].

A procedure 920 for returning the fine path W_{fine} for the signals X and Y uses the information received in step 902 and calculates the resampled raw signal X′ in step 922, similar to step 520 in FIG. 5. In step 924, J seeds {Qᵢ} are generated using the function SelectSeeds(⋅) The SelectSeeds(⋅) is the procedure that gets J segments with a length of K from the query signal Y. In step 926, the final end point to is calculated for each seed Qᵢ, using the procedure DSDTW discussed in step 510 in FIG. 5.

In step 928, a maximal level coarsening ratio L is calculated and in step 930 the CWT for the resampled raw current signal X′ and for the query signal Y are calculated. Then, in step 932, the coarse path W_{coarse} for the CWT of the resampled raw signal and the query signal is calculated using the function CoarsePath. This function is defined as follow: (1) for a given query Y having a length M, calculate the maximal level coarsening ratio a as being given by: a=log₂ (M)−2, (2) obtain the feature signal for both CWT(X′, a) and CWT(Y,a), (3) run the direct subsequence DTW distance on the feature signals from the X′ and Y and determine all the possible paths W_{I}, (4) find out the coarse path W_{I} that covers the seeds Wᵢ, and (5) combine both the seeds and the coarse path to generate a more detailed path W_{coarse}.

Returning to the cwSDTWnano method 900, in step 934 the path Wi is calculated using the cwDTW(⋅) function, which is the continuous wavelet-based multi-level dynamic time warping discussed in [5]. Note that this function calculates the path Wi based on the resampled raw signal X′, the query signal Y, and the coarse path W_{coarse} calculated in step 932. In step 936, an improved path B is calculated based on the path W_{I}, using the function ReMapindex(⋅) which is a context-dependent constraint generation from a coarse path W_{coarse} with a window size of r, similar to the function discussed in step 528 in FIG. 5. Then, in step 938, the fine path W_{fine} is calculated by applying the function cDTW(⋅), which is the constrained dynamic time warping [20], to the raw signal X, the query Y, and the path B.

It is noted that the false filtering procedure, which was discussed with regard to FIG. 8 and implemented in steps 922 to 926 in FIG. 9, has a complexity of

$$O ( \frac{N}{{s}_{b a s e}} K J )$$

and the cDTW(⋅) procedure is bounded within O(NlogN), so that the overall complexity for the method 900 is

$$O ( \frac{N}{{s}_{b a s e}} K J + N log N ) ,$$

which is advantageous when the signal length grows.

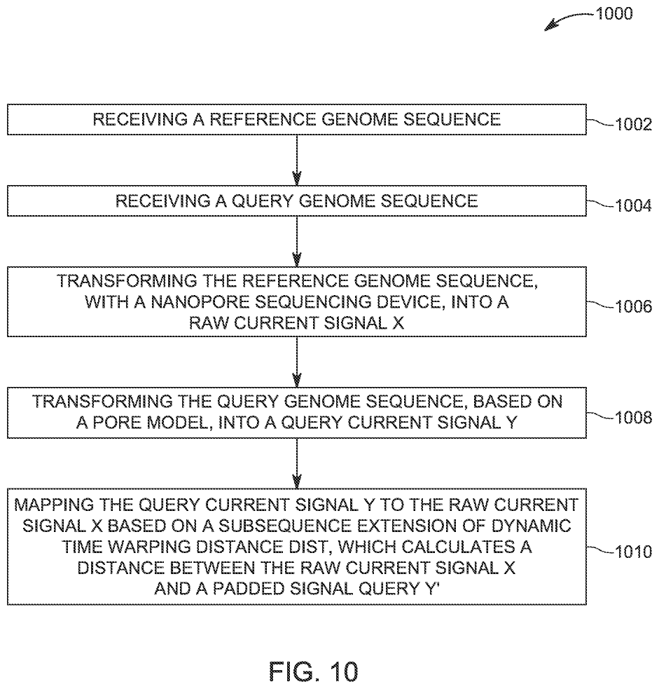

The two novel methods DSDTWnano and cwSDTWnano discussed above can be summarized as follows. The DSDTWnano method 1000 is a method for similarity searching directly on nanopore raw current signals and is illustrated in FIG. 10. The method includes a step 1000 of receiving a reference genome sequence, a step 1002 of receiving a query genome sequence, a step 1004 of transforming the reference genome sequence, with a nanopore sequencing device, into a raw current signal X, a step 1006 of transforming the query genome sequence, based on a pore model, into a query current signal Y, and a step 1008 of mapping the query current signal Y to the raw current signal X based on a subsequence extension of dynamic time warping distance Dist, which calculates a distance between the raw current signal X and a padded signal query Y′. The padded signal query Y′ is the query current signal Y to which an element y₀ has been added, the raw current signal X and the query current signal Y are electrical currents, and the raw current signal X corresponds to a genome of an organism.

In one embodiment, the method may further include resampling the raw current signal X to obtain a resampled raw current signal X′, and/or calculating the distance between the raw current signal X and the padded signal query Y′ by using the resampled raw current signal X′, and/or calculating an end point tₑ between a similarity of the raw current signal X and the query current signal Y, and calculating a coarse path W_{coarse} based on the end point tₑ, the resampled raw current signal X′, and the query current signal Y, where the coarse path W_{coarse} is a list that maps elements of the raw current signal X to the query current signal Y.

The method may further include a step of calculating a fine path W_{fine} based on the coarse path W_{coarse}, the raw current signal X, and the query current signal Y, where the fine path W_{fine} is a list that maps the query current signal Y to the raw current signal X with higher accuracy than the coarse path W_{coarse}. A length of the query genome sequence is at least 128.

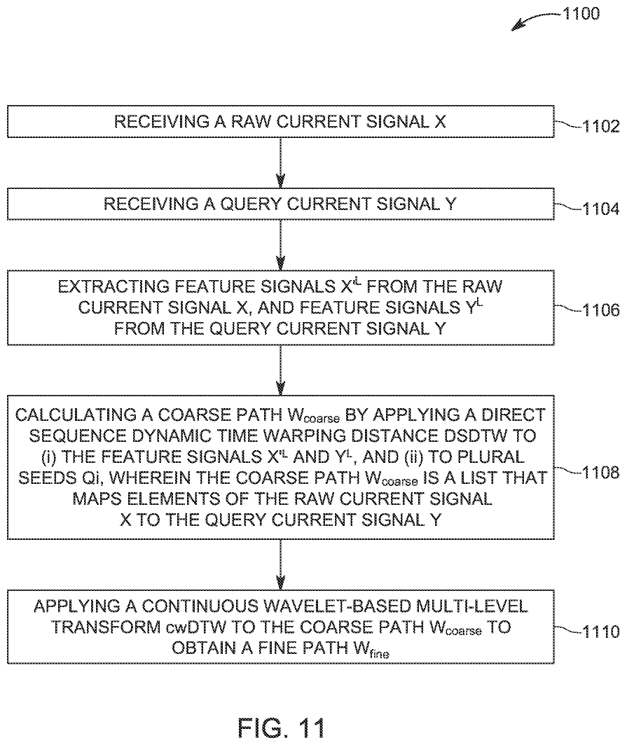

The DSDTWnano method 1100 is a method for similarity searching directly on nanopore raw current signals and is illustrated in FIG. 11. The method includes a step 1100 of receiving a raw current signal X, a step 1102 of receiving a query current signal Y, a step 1104 of extracting feature signals X′^{L} from the raw current signal X, and feature signals Y^{L} from the query current signal Y, a step 1106 of calculating a coarse path W_{coarse} by applying a direct sequence dynamic time warping distance DSDTW to (i) the feature signals X′^{L} and Y^{L}, and (ii) to plural seeds Q, wherein the coarse path W_{coarse} is a list that maps elements of the raw current signal X to the query current signal Y, and a step 1108 of applying a continuous wavelet-based multi-level transform cwDTW to the coarse path W_{coarse} to obtain a fine path W_{fine}. The fine path W_{fine} is a list that more accurately maps elements of the raw current signal X to the query current signal Y, then the coarse path W_{coarse}.

The method may further include a step of extracting the feature signals from a transformed spectrum CWT(X′) of a resampled raw current X′, which corresponds to the raw current signal X, and a step of extracting the feature signals from a transformed spectrum CWT(Y) of the query current signal Y, where the transformed spectrum is obtained with a continuous wavelet transform.

The method may also include a step of receiving a reference genome sequence, a step of receiving a query genome sequence, a step of transforming the reference genome sequence, with a nanopore sequencing device, into the raw current signal X, and a step of transforming the query genome sequence, based on a pore model, into the query current signal Y. The method may also include a step of calculating a maximal level coarsening ratio a for the query current signal Y, calculating the feature signals based on the maximal level coarsening ratio a of the query current signal Y and a continuous wavelet transform CWT of a resampled raw current signal X′, which corresponds to the raw current signal X, and calculating the feature signals based on the maximal level coarsening ratio a of the query current signal Y and a continuous wavelet transform CWT of the query current signal Y. In one application, the direct sequence dynamic time warping distance DSDTW is applied to the resampled raw circuit signal X′ and the query current signal Y.

The two methods discussed above have been tested by the inventors on real nanopore datasets for both their accuracy and speed as now discussed. Three nanopore sequencing datasets were used in the experiments, among which two nanopore sequencing datasets are used to demonstrate the effectiveness of the two novel methods for similarity search accuracy and one of the datasets is used to demonstrate the potential application of the novel methods to SNP detection.

The first dataset is a subset of the publicly available Human data (Human21 database). This dataset comes from Human chromosome 21 from the Nanopore WGS Consortium. The samples in this dataset were sequenced from the NA12878 human genome reference on the Oxford Nanopore MinION using 1D ligation kits (450 bp/s) with R9.4 flow cells.

The second dataset is the genome of Lambda phage (Lambda phage database), which was prepared and sequenced at the University of Queensland. The samples were sequenced on the MinION device with 1D protocol on R9.4 flow cells (FLO-MIN106 protocol).

The third dataset is the genome of E. coli (E. coli database), which was also prepared and sequenced at the University of Queensland on the MinION device with 1D protocol on R9.4 flow cells (FLO-MIN106 protocol). This dataset is used in this discussion for the study of the SNP detection.

To verify the novel methods 1000 and 1100, the query signal was selected with different lengths and the raw current signals were selected with different lengths. In one application, the test selected different small random segments of DNA sequences that range from 1,000˜5,000 lengths and then the two methods were used to detect the selected DNA segments on the raw current signals. In another application, the test selected a random length-fixed segment of DNA sequence for each read and then the methods were used to detect the selected DNA segment on the raw current signals with different lengths that range from 50,000 to about 200,000.

Because the exact mapping between the DNA sequence and the raw current signal has already been known for HM4530 and PP4782, for these databases, the exact answer is available for the mapping between the DNA sequence and the raw current signal, as well as the accurate locations of the mapping. For a query genome G given by G=g₁g₂ . . . g_{L}, the accuracy of the query result W can be measured based on the mapping difference (genome-to-signal location difference on each nucleic acid) between the query answer and the ground-truth (named as edit mapping error), which is defined as:

$$e m E r r o r ( W ) = \frac{1}{L} {\sum }_{i = 1}^{L} \frac{E d i t D i s t ( s i g n a {l}_{q} ( {g}_{i} ) , s i g n a {l}_{g t} ( {g}_{i} ) )}{L ( s i g n a {l}_{g t} ( {g}_{i} ) )} , \qquad ( 13 )$$

where signal_{q}(gᵢ) returns the signal (or “event”) indexes corresponding to the gᵢ on the nanopore raw current signal of the query result, signal_{gt}(gᵢ) returns the signals indexes corresponding to the gᵢ on the nanopore raw current signal of groundtruth, EditDist(⋅) is the edit distance between two strings, and L(⋅) is the length of the signal. In this application, the analysis is based on the query DNA sequence, but not the raw current signal. For example, for a query G=g₁g₂g₃ with L=3, and a query answer of {10, 11, 12, 13, 14, 15, 16, 17}-th items on the raw current signal with g₁ corresponding to {10, 11, 12}-th signals, g₂ corresponding to {13, 14, 15}-th signals, and g₃ corresponding to {16, 17}-th signals, where the groundtruth is the {11, 12, 13, 14, 15, 16, 17}-th items on the raw current signal with g₁ corresponding to {11, 12}-th signals while the other two being the same to those of the query answer, the EditDist (signal_{q}(gᵢ),signal_{gt}(gᵢ))=1 and emError(W)=⅓·(½+ 0/3+ 0/2)=0166. If the algorithm returns a perfect warping path, the error is zero. Note that the error may exceed 100% if the distance of the warping path is completely wrong.

While the above experiment used the groundtruth to determine the accuracy of the novel methods, the present embodiment is evaluating their accuracies without this information. Under these circumstances, the normalized signal distance nDist(W) of a warping path W=w₁, w₂, . . . , wₖ is proposed for further data filtering and clustering, and the normalized signal distance is defined as:

$$n D i s t ( W ) = \frac{{\sum }_{n = 1}^{K} c ( {w}_{ni} , {w}_{n j} )}{K} , \qquad ( 14 )$$

where K is the length of the warping path and c(wₙᵢ, wₙⱼ) is the absolute distance of the nth aligned element between the two signal points xᵢ (from the nanopore signal X) and yⱼ (which is translated from the DNA sequence according to the k-mer model). Different from the emError(W) introduced in equation (13), the nDist(W) is defined as the difference of the signals and not of the mapping indexes.

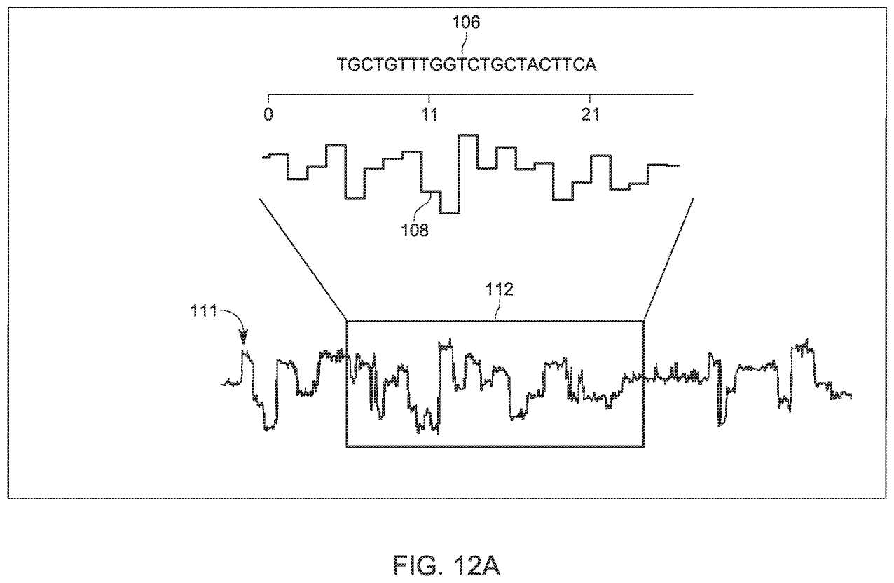

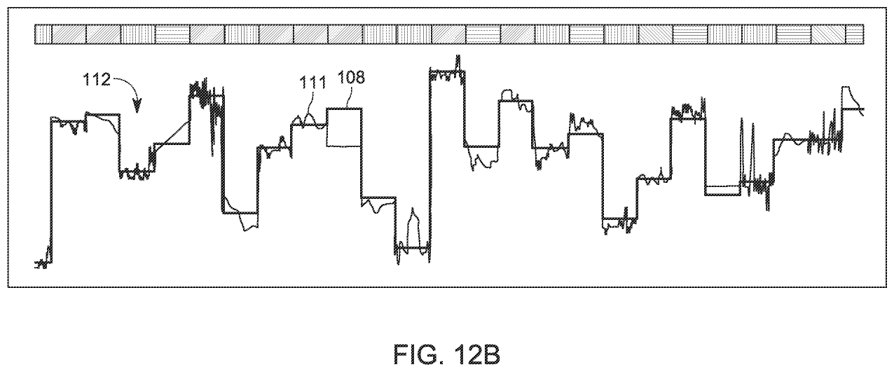

Because the genome-to-signal search is not intuitive, a realistic example is presented to demonstrate the effectiveness of the methods discussed herein, in discovering the qualifying subsequences. FIGS. 12A and 12B illustrate such an example, which is a short segment (locate) of a read query that was selected from the Human chromosome 21 dataset. A short read 106 (500-long) is selected as the query and a raw signal 111 (106,960 long) is selected as the searching database. Firstly, the query DNA subsequence 106 is translated into a query signal 108 based on the 6-mer pore model provided by the Nanopore Technology, as illustrated in FIG. 12A. Then, the DSDTWnano method was run for these signals and the detailed region 112 was obtained for the nanopore raw current signal 111, which has the maximal similarity with the query signal 108, as illustrated at the bottom of FIG. 12A. This query search took 197 ms and resulted in a normalized signal distance of 0.1556.

Typically, a normalized signal distance range from 0˜1.8 indicates a query response with a high-level similarity. FIG. 12B shows the detailed current change that locates between 300 to 400 on the query signal, and also shows the mapping area on the raw signal 111 that corresponds to the segments shows in FIG. 12A. It can be noted that the details of these two signal segments are very similar to each other, which give out the direct visual confidence of the novel method 1000.

The inventors have further investigated the performance of the DSDTWnano and cwSDTWnano methods on the Human21 database and Lambda phage database. For these two datasets, the inventors randomly sampled 3000 reads from each dataset. The average length of the DNA sequences in the sampled dataset is 7,749 and 6,928 for the Human and Lambda phage, respectively, whereas the average length of the nanopore raw current signal sequences is 85,102, and 59,012 for the Human and Lambda phage, respectively. The temporal scale ratio between the nanopore raw current signal sequence and the corresponding DNA sequence is around 9. For the Human21 database and Lambda phage database, the inventors prepared the genome-to-signal mapping of each read based on their base-calling and a dynamic programming based end-to-end refinement. These genome-to-signal mapping serves as the ground-truth information that describes the index-to-index relationship between a read and its nanopore raw current signal.

For the genome-to-signal similarity search, the inventors randomly selected a segment with length/as the query and then the DSDTWnano and cwSDTWnano methods were run on the corresponding raw current signal to find out its maximal response mapping (in this experiments, the cwSDTWnano method was run with K=3 and L=128). Then the query results generated by the DSDTWnano and cwSDTWnano methods were compared to the groundtruth using the edit mapping error emError(W) defined by equation (13).

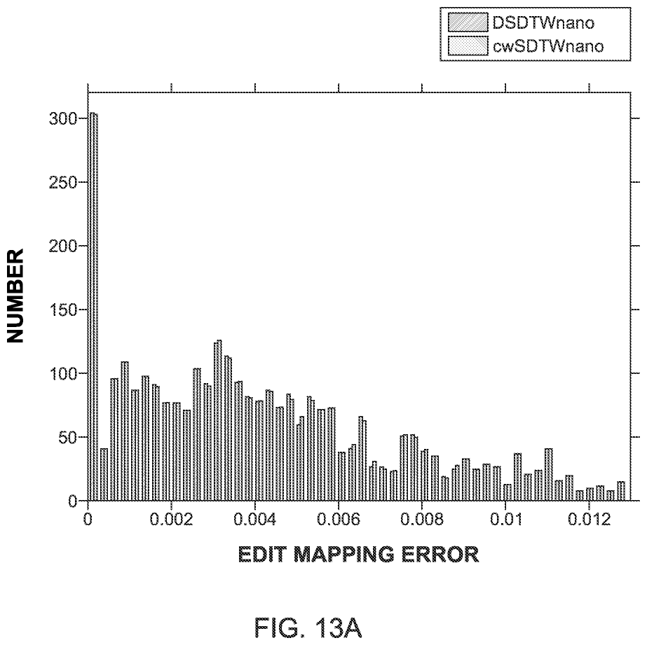

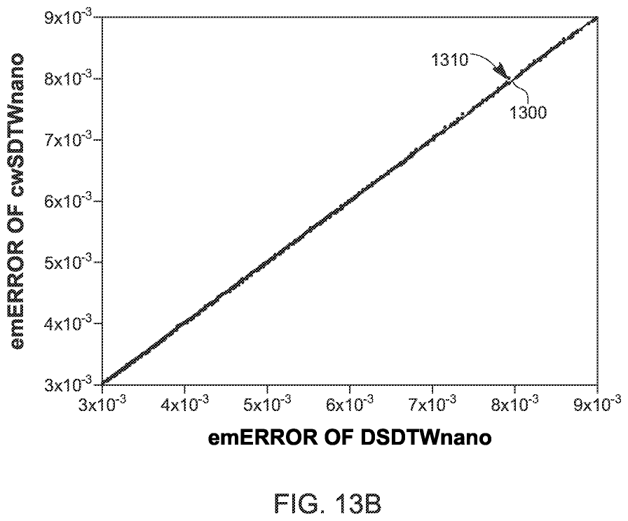

FIG. 13A shows the statistics of the editor mapping error emError(W) related to the DSDTWnano and cwSDTWnano methods when applied with the mapping boundary r=50 and query length I=1,000. The distributions of the edit mapping error emError(W) for the DSDTWnano and cwSDTWnano methods are shown in FIG. 13A. It is observed that the edit mapping error emError(W) of the DSDTWnano and cwSDTWnano methods are very close to each other and the average of the edit mapping error is in the range from 0 to 0.01, which is a very small error value. FIG. 13A also indicates that the DSDTWnano method produces better results than the cwSDTWnano method. The error values between the output of the DSDTWnano method and the cwSDTWnano method for each read are substantially the same, as illustrated by line 1300 in FIG. 13B. It is observed that most of these values are the same (on the diagonal of the scatter map), but some of these values 1310 deviate from the optimum line 1300. The degeneration of the cwSDTWnano method's results may be caused by the coarsening in the algorithm. However, the cwSDTWnano method is about 2× to 5× faster than the DSDTWnano method and still produces very small editor mapping errors. Therefore, the cwSDTWnano method is recommended if the available computational resources are limited.

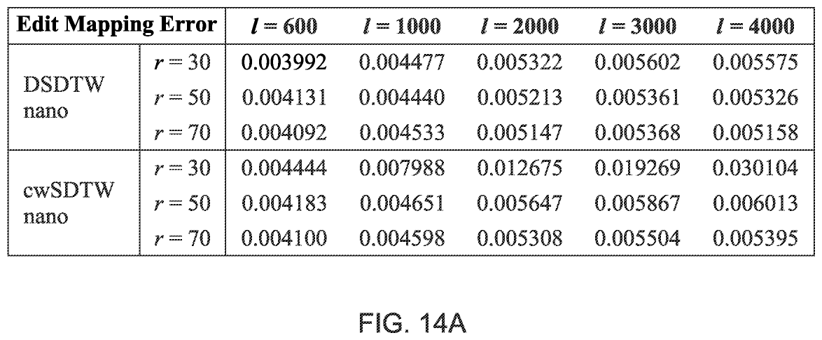

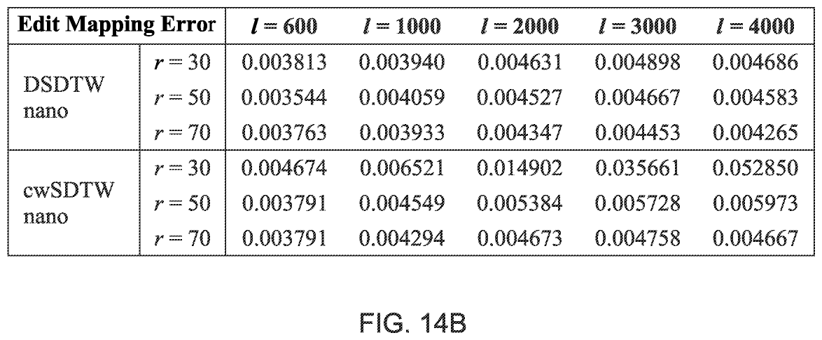

Furthermore, the inventors have tested both the DSDTWnano and cwSDTWnano methods with queries of different lengths l and different radius of the mapping path boundary r. The average edit mapping error of the query results obtained by using the DSDTWnano and cwSDTWnano methods when applied to the Human21 database and Lambda phage database is summarized in Table 1 and Table 2, respectively, which are shown in FIGS. 14A and 14B. Based on Table 1 and Table 2, it is noted that for queries with different lengths, the DSDTWnano method always outputs a query result within a 0.01 edit mapping error. For most cases, the average edit mapping error is no larger than 0.006. The performance of the cwSDTWnano method depends on the mapping boundary r. From Table 1 and Table 2 it is noted that the edit mapping error for the cwSDTWnano method is around 0.006 if a suitable r is selected (r=50 for l≤2000 and r=70 for l≤4000). Although the Human and Lambda phage databases correspond to totally different species, the small differences between the results of the Table 1 and Table 2 suggest that the results of the novel methods will not change substantially for other species.

Because the edit mapping error directly reflects the mapping error related to the nucleic acid positions, it can also indicate the necessary signal coverage (an analogy to the coverage of reads) to ensure the further success of clustering or mutation analysis. A small edit mapping error means that there is little theoretical error introduced by the algorithm. Specifically, because the edit mapping error is smaller than 1% in the tables, it can be inferred that a 10×˜20× signal coverage is enough for a further mutation analysis, if the raw signals are searched and mapped by the novel algorithms presented in FIGS. 5 and 9.

For a database with a number of potential raw current signals, the time necessary for performing a query task is significant, as there is a general expectation that this time is minimal. Generally, the runtime of the DSDTWnano algorithm is about 450 ms and that of the cwSDTWnano algorithm is about 200 ms for a query with 1,000 length on a 100,000-long raw current signal. When the query length grows, the runtime may increase to 1˜2 seconds, which becomes a considerable time cost if there are hundreds or thousands of raw current signals to be considered. Under these conditions, the cwSDTWnano method is best suited because it can accelerate the query process by using the multi-scale schedule.

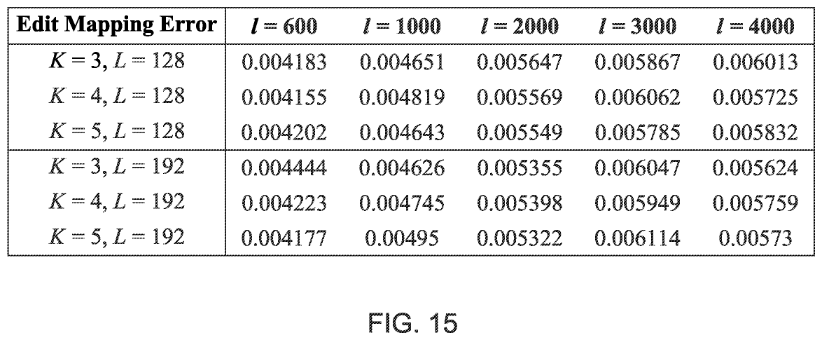

The runtime for both the DSDTWnano and cwSDTWnano methods is now discussed. Because the cwSDTWnano method has the extra parameters of the seed number K and seed length L to define, the accuracy of the cwSDTWnano method under different configurations on the Human21 database was considered. Table 3 in FIG. 15 summarizes the average edit mapping error of the query results on the Human21 database for the cwSDTWnano method, when considered with different seed numbers K and seed lengths L (here the search radius r is set to 70). Based on Table 3, it is noted that the seed length L influences the quality of the result, but the seed number K does not. These results may be caused by the good coarsening function (continuous wavelet) used in the cwSDTWnano method, which preserves the major features under a given scale. From the edit mapping error illustrated in Table 3, it is concluded that the cwSDTWnano method is robust for K≥3 and L≥128. The experiments run for this method with the parameters illustrated in Table 3 indicated that (i) a too short seed may cause false dismissals, (ii) a seed length of 128 can ensure the correctness of the cwSDTWnano determinations for most of the queries, and (iii) a seed length of 192 is enough for a dataset with qualified raw current signals. The different choices of the searching parameters r, K, and L will also affect the runtime. However, because these parameters affect linearly the execution of the two methods 1000 and 1100, their influence on the runtime can be inferred by the changing ratios.

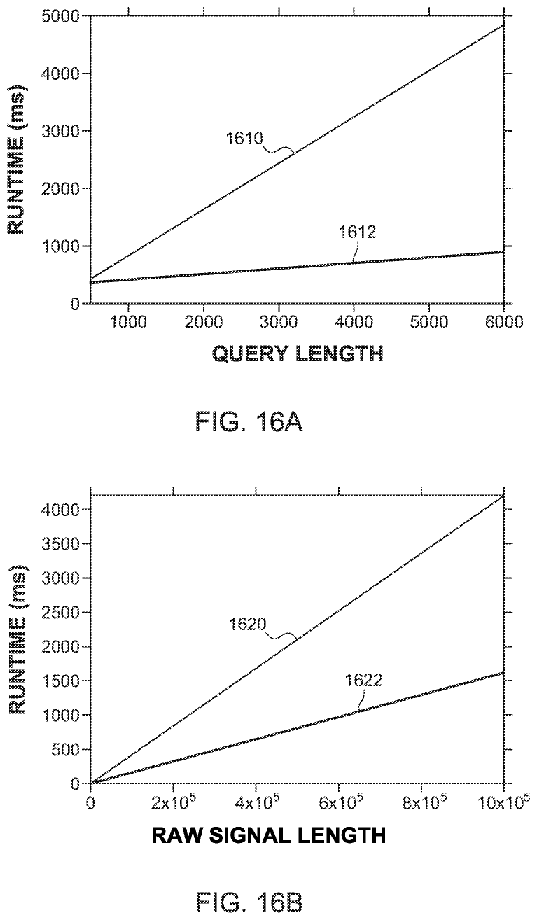

In this regard, FIG. 16A shows the runtime 1610 for the DSDTWnano method and the runtime 1612 for the cwSDTWnano method for different query lengths and FIG. 16B shows the runtime 1620 for the DSDTWnano method and the runtime 1622 for the cwSDTWnano method for searches using various raw current signal lengths. The execution times in FIGS. 16A and 16B were calculated on a Fedora25 system with 128 Gb memory and two E5-2667v4 (3.2 GHz) processors. It is observed from the figures that the collected data follows a near-linear relationship (according to the theoretical complexity of the DSDTWnano and cwSDTWnano methods, the DSDTWnano method's cost increases linearly with the change of query length and raw signal length, and the cwSDTWnano method's cost increases following a O(N+log(N)) complexity). FIG. 16A shows that the DSDTWnano method has a much higher execution time as the query length increases. On the contrary, the cwSDTWnano method maintains a low computational cost. It is noted that the runtime of the cwSDTWnano method is always smaller than 900 ms, even when searching a 6,000-long query on a raw signal with a 2×10⁵ length. It is further observed, based on FIG. 16A, that the runtime of the cwSDTWnano method does not exceed 1,500 ms when searching a 1,000-long query on a raw current signal having a 1×10⁶ length.

Therefore, the introduction of the coarsening analysis by the CWT causes a degeneration in the result of the cwSDTWnano method (as illustrated in Table 1 and Table 2), but the loss of the accuracy is acceptable when considering the benefit of the shortened execution speed. Thus, for a given database, it is possible to (i) run the DSDTWnano method if the query length is short, and (ii) run the cwSDTWnano method if the query is long.

Single nucleotide polymorphisms (SNPs) is a variation in a single nucleotide that occurs at a specific position in the genome. The SNPs were found to be involved in the etiology of many human diseases and are also critical for personalized medicine. Currently, the identification of the SNPs is mainly achieved by resequencing approach (i.e., searching for differences between aligned reads and the reference genome) or assembly approach (i.e., de novo assemble consensus read sequences against a reference genome). However, few works explored the capability of nanopore sequencing to identify SNPs. Reports showed that to reach high detection rate (such as >95%), it is need more than 60× sequencing coverage.

In this embodiment, a novel approach to identify and visualize SNPs using nanopore signal data at a relatively low coverage (20×) is discussed. First, a synthetic reference genome is generated by randomly substituting 100 bases on the sense strand of the E. coli reference genome. Then, a 2,000-long sequence is extracted from the synthetic genome that might contain a SNP, and this reference sequence is used as a query against the nanopore signal database by using the cwSDTWnano method to localize the candidate raw signal segments and positions that might contain the SNP. These candidate positions are chosen based on the mismatches between the aligned signals and the reference sequence's k-mer pore model. Afterwards, for each candidate position, four genomic sequences with length 600, centered at this position with four possible nucleotide {A, T, C, G} are selected as the query sequences to search against the signal database again. Note that in this embodiment the method filters out those signals whose normalized distance (see equation (14)) is larger than a given threshold (by default the threshold is 0.18) to the standard signal that translated from the query sequence (based on the k-mer pore model). Finally, the mutation (nucleotide X, Xϵ{A, T, C, G}) with an expected current signal closest to the observed signals in the database is chosen as the possible SNP at the candidate position.

The novelty of the cwSDTWnano algorithm for SNP detection resides in the position-specific signal-based search strategy, which is different from those read-based strategies. Although the base-calling error in the nanopore sequencing is around 15%, and the signal quality also varies over different regions in a single read, the novel cwSDTWnano method is not negatively affected by these effects due to the following two facts: (i) the normalized signal distance measurement guarantees the success in filtering out the low-quality signals, and (ii) the low theoretical error (which have been proved in the edit mapping error analysis above) renders the remaining signals to have a high mapping accuracy and enables the algorithm to work with a relative low-signal coverage. Thus, with the guarantee of signal quality, the novel method is able to take as input the position-specific query region and to search against the signal database for identifying the SNPs, which is more intuitive and direct when compared to the traditional read-based strategies.

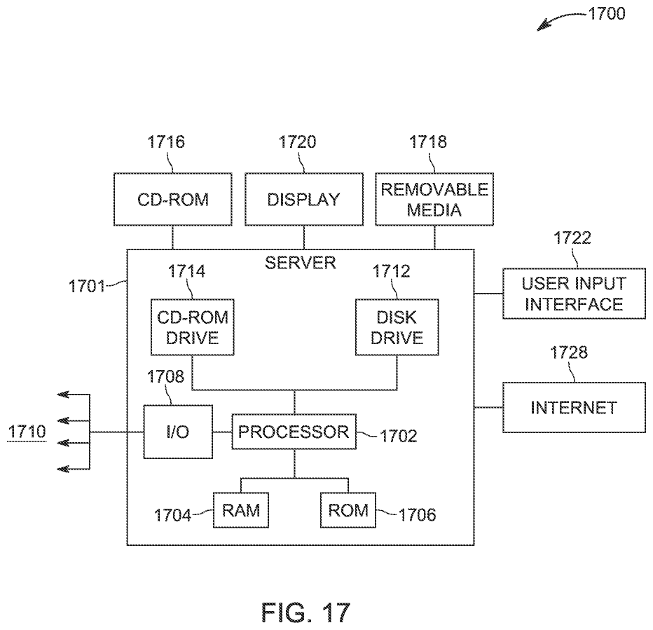

The above-discussed procedures and methods may be implemented in a computing device as illustrated in FIG. 17. Hardware, firmware, software or a combination thereof may be used to perform the various steps and operations described herein.

Exemplary computing device 1700 suitable for performing the activities described in the exemplary embodiments may include a server 1701. Such a server 1701 may include a central processor (CPU) 1702 coupled to a random access memory (RAM) 1704 and to a read-only memory (ROM) 1706. ROM 1706 may also be other types of storage media to store programs, such as programmable ROM (PROM), erasable PROM (EPROM), etc. Processor 1702 may communicate with other internal and external components through input/output (I/O) circuitry 1708 and bussing 1710 to provide control signals and the like. Processor 1702 carries out a variety of functions as are known in the art, as dictated by software and/or firmware instructions.

Server 1701 may also include one or more data storage devices, including hard drives 1712, CD-ROM drives 1714 and other hardware capable of reading and/or storing information, such as DVD, etc. In one embodiment, software for carrying out the above-discussed steps may be stored and distributed on a CD-ROM or DVD 1716, a USB storage device 1718 or other form of media capable of portably storing information. These storage media may be inserted into, and read by, devices such as CD-ROM drive 1714, disk drive 1712, etc. Server 1701 may be coupled to a display 1720, which may be any type of known display or presentation screen, such as LCD, plasma display, cathode ray tube (CRT), etc. A user input interface 1722 is provided, including one or more user interface mechanisms such as a mouse, keyboard, microphone, touchpad, touch screen, voice-recognition system, etc.

Server 1701 may be coupled to various devices, for example, sequencing device 120. The server may be part of a larger network configuration as in a global area network (GAN) such as the Internet 1728, which allows ultimate connection to various landline and/or mobile computing devices.

The disclosed embodiments provide direct subsequence search algorithms, DSDTWnano and cwSDTWnano, for the genome-to-signal similarity search and mapping. The proposed algorithms are based on the idea of Dynamic Time Warping and directly operate on the nanopore raw current signals without any loss of information in data translation. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] Deamer, D., Akeson, M., and Branton, D. (2016). Three decades of nanopore sequencing. Nature biotechnology, 34(5), 518.
[2] Rang, F. J., Kloosterman, W. P., and de Ridder, J. (2018). From squiggle to basepair: computational approaches for improving nanopore sequencing read accuracy. Genome biology, 19(1), 90.
[3] Rand, A. C., Jain, M., Eizenga, J. M., Musselman-Brown, A., Olsen, H. E., Akeson, M., and Paten, B. (2017). Mapping dna methylation with high-throughput nanopore sequencing. nature methods, 14(4), 411-413.
[4] Stoiber, M. H., Quick, J., Egan, R., Lee, J. E., Celniker, S. E., Neely, R., Loman, N., Pennacchio, L., and Brown, J. B. (2016). De novo identification of dna modifications enabled by genome-guided nanopore signal processing. bioRxiv, page 094672.
[5] Han, R., Li, Y., Gao, X., and Wang, S. (2018). An accurate and rapid continuous wavelet dynamic time warping algorithm for end-to-end mapping in ultra-long nanopore sequencing. Bioinformatics, 34(17), i722-i731.
[6] Gollery, M. (2005). Bioinformatics: Sequence and genome analysis. Clinical Chemistry, 51(11), 2219-2219.
[7] Berndt, D. J. and Clifford, J. (1996). Finding patterns in time series: a dynamic programming approach. In Advances in knowledge discovery and data mining, pages 229-248. American Association for Artificial Intelligence.
[8] Keogh, E. and Ratanamahatana, C. A. (2005). Exact indexing of dynamic time warping. Knowledge and information systems, 7(3), 358-386.
[9] Al-Naymat, G., Chawla, S., and Taheri, J. (2009). Sparsedtw: A novel approach to speed up dynamic time warping. In Proceedings of the Eighth Australasian Data Mining Conference-Volume 101, pages 117-127. Australian Computer Society, Inc.
[10] Salvador, S. and Chan, P. (2007). FastDTW: Toward accurate dynamic time warping in linear time and space. Intelligent Data Analysis, 11(5), 561-580.
[11] Muller, M., Mattes, H., and Kurth, F. (2006). An efficient multiscale approach to audio synchronization. In ISMIR, pages 192-197.
[12] Pratzlich, T., Driedger, J., and Muller, M. (2016). Memory-restricted multiscale dynamic time warping. In Acoustics, Speech and Signal Processing (ICASSP), 2016 IEEE International Conference on, pages 569-573. IEEE.
[13] Chen, Y., Nascimento, M. A., Ooi, B. C., and Tung, A. K. (2007). Spade: On shape-based pattern detection in streaming time series. In Data Engineering, 2007. ICDE 2007. IEEE 23rd International Conference on, pages 786-795. IEEE.
[14] Zhou, M. and Wong, M. H. (2008). Efficient online subsequence searching in data streams under dynamic time warping distance. In Data Engineering, 2008. ICDE 2008. IEEE 24th International Conference on, pages 686-695. IEEE.
[15] Sakurai, Y., Faloutsos, C., and Yamamuro, M. (2007). Stream monitoring under the time warping distance. In Data Engineering, 2007. ICDE 2007. IEEE 23rd International Conference on, pages 1046-1055. IEEE.
[16] Berlin, K., Koren, S., Chin, C.-S., Drake, J. P., Landolin, J. M., and Phillippy, A. M. (2015). Assembling large genomes with single-molecule sequencing and locality-sensitive hashing. Nature biotechnology, 33(6), 623.
[17] Jain, C., Koren, S., Dilthey, A., Phillippy, A. M., and Aluru, S. (2018a). A fast adaptive algorithm for computing whole-genome homology maps. Bioinformatics, 34(17), i748-i756.
[18] Li, H. (2018). Minimap2: pairwise alignment for nucleotide sequences. Bioinformatics, 1, 7.
[19] Saramaki, T. and Bregovic, R. (2002). Multirate systems and filterbanks. In Multirate systems: design and applications, pages 27-85. IGI Global.
[20] Ratanamahatana, C. A. and Keogh, E. (2005). Three myths about dynamic time warping data mining. In Proceedings of the 2005 SIAM International Conference on Data Mining, pages 506-510. SIAM.
[21] Mallat, S. G. (1989). A theory for multiresolution signal decomposition: the wavelet representation. IEEE transactions on pattern analysis and machine intelligence, 11(7), 674-693.

1. A method for similarity searching directly on nanopore raw current signals, the method comprising:
receiving a reference genome sequence;
receiving a query genome sequence;
transforming the reference genome sequence, with a nanopore sequencing device, into a raw current signal X;
transforming the query genome sequence, based on a pore model, into a query current signal Y; and
mapping the query current signal Y to the raw current signal X based on a subsequence extension of dynamic time warping distance Dist, which calculates a distance between the raw current signal X and a padded signal query Y′,
wherein the padded signal query Y′ is the query current signal Y to which an element y₀ has been added,
wherein the raw current signal X and the query current signal Y are electrical currents, and
wherein the raw current signal X corresponds to a genome of an organism.

2. The method of claim 1, further comprising:
resampling the raw current signal X to obtain a resampled raw current signal X′.

3. The method of claim 2, further comprising:
calculating the distance between the raw current signal X and the padded signal query Y′ by using the resampled raw current signal X′.

4. The method of claim 3, further comprising:
calculating an end point tₑ between a similarity of the raw current signal X and the query current signal Y; and
calculating a coarse path W_{coarse} based on the end point tₑ, the resampled raw current signal X′, and the query current signal Y,
wherein the coarse path W_{coarse} is a list that maps elements of the raw current signal X to the query current signal Y.

5. The method of claim 4, further comprising:
calculating a fine path W_{fine} based on the coarse path W_{coarse}, the raw current signal X, and the query current signal Y,
wherein the fine path W_{fine} is a list that maps the query current signal Y to the raw current signal X with higher accuracy than the coarse path W_{coarse}.

6. The method of claim 1, wherein a length of the query genome sequence is at least 128.

7. A computing device for similarity searching directly on nanopore raw current signals, the device comprising:
an input/output interface configured to receive a reference genome sequence and a query genome sequence; and
a processor connected to the input/output interface and configured to,
transform the reference genome sequence, with a nanopore sequencing device, into a raw current signal X,
transform the query genome sequence, based on a pore model, into a query current signal Y, and
map the query current signal Y to the raw current signal X based on a direct subsequence dynamic time warping distance Dist, which is configured to calculate a distance between the raw current signal X and a padded signal query Y′,
wherein the padded signal query Y′ is the query current signal Y to which an element y₀ has been added,
wherein the raw current signal X and the query current signal Y are electrical currents, and
wherein the raw current signal X corresponds to a genome of an organism.

8. The device of claim 7, wherein the processor is further configured to:
resample the raw current signal X to obtain a resampled raw current signal X′.

9. The device of claim 8, wherein the processor is further configured to:
calculate the distance between the raw current signal X and the padded signal query Y′ by using the resampled raw current signal X′.

10. The device of claim 9, wherein the processor is further configured to:
calculate an end point tₑ between a similarity of the raw current signal X and the query current signal Y; and
calculate a coarse path W_{coarse} based on the end point tₑ, the resampled raw current signal X′, and the query current signal Y,
wherein the coarse path W_{coarse} is a list that maps elements of the raw current signal X to the query current signal Y.

11. The device of claim 10, wherein the processor is further configured to:
calculate a fine path W_{fine} based on the coarse path W_{coarse}, the raw current signal X, and the query current signal Y,
wherein the fine path W_{fine} is a list that maps the query current signal Y to the raw current signal X with higher accuracy than the coarse path W_{coarse}.

12. The device of claim 7, wherein a length of the query genome sequence is at least 128.

13. A method for similarity searching directly on nanopore raw current signals, the method comprising:
receiving a raw current signal X;
receiving a query current signal Y;
extracting feature signals X′^{L} from the raw current signal X, and feature signals Y^{L} from the query current signal Y;
calculating a coarse path W_{coarse} by applying a direct sequence dynamic time warping distance DSDTW to (i) the feature signals X′^{L} and Y^{L}, and (ii) to plural seeds Qᵢ, wherein the coarse path W_{coarse} is a list that maps elements of the raw current signal X to the query current signal Y; and
applying a continuous wavelet-based multi-level transform cwDTW to the coarse path W_{coarse} to obtain a fine path W_{fine},
wherein the fine path W_{fine} is a list that more accurately maps elements of the raw current signal X to the query current signal Y, then the coarse path W_{coarse}.

14. The method of claim 13, further comprising:
extracting the feature signals X′^{L} from a transformed spectrum CWT(X′) of a resampled raw current X′, which corresponds to the raw current signal X; and
extracting the feature signals Y^{L} from a transformed spectrum CWT(Y) of the query current signal Y,
wherein the transformed spectrum is obtained with a continuous wavelet transform.

15. The method of claim 13, further comprising:
receiving a reference genome sequence;
receiving a query genome sequence;
transforming the reference genome sequence, with a nanopore sequencing device, into the raw current signal X; and
transforming the query genome sequence, based on a pore model, into the query current signal Y.

16. The method of claim 13, further comprising:
calculating a maximal level coarsening ratio a for the query current signal Y;
calculating the feature signals X′^{L} based on the maximal level coarsening ratio a of the query current signal Y and a continuous wavelet transform CWT of a resampled raw current signal X′, which corresponds to the raw current signal X; and
calculating the feature signals Y^{L} based on the maximal level coarsening ratio a of the query current signal Y and a continuous wavelet transform CWT of the query current signal Y.

17. The method of claim 16, wherein the direct sequence dynamic time warping distance DSDTW is applied to the resampled raw circuit signal X′ and the query current signal Y.

18. A computing device for similarity searching directly on nanopore raw current signals, the computing device comprising:
an input/output interface configured to receive a raw current signal X and to receive a query current signal Y; and
a processor connected to the input/output interface and configured to,
extract feature signals X′^{L} from the raw current signal X, and feature signals Y^{L} from the query current signal Y,
calculate a coarse path W_{coarse} by applying a direct sequence dynamic time warping distance DSDTW to (i) the feature signals X′^{L} and Y^{L}, and (ii) to plural seeds Qᵢ, wherein the coarse path W_{coarse} is a list that maps elements of the raw current signal X to the query current signal Y, and
apply a continuous wavelet-based multi-level transform cwDTW to the coarse path W_{coarse} to obtain a fine path W_{fine},
wherein the fine path W_{fine} is a list that more accurately maps elements of the raw current signal X to the query current signal Y, then the coarse path W_{coarse}.

19. The computing device of claim 18, wherein the processor is further configured to:
extract the feature signals X′^{L} from a transformed spectrum CWT(X′) of a resampled raw current X′, which corresponds to the raw current signal X; and
extract the feature signals Y^{L} from a transformed spectrum CWT(Y) of the query current signal Y,
wherein the transformed spectrum is obtained with a continuous wavelet transform.

20. The computing device of claim 18, wherein the processor is further configured to:
calculate a maximal level coarsening ratio a for the query current signal Y;
calculate the feature signals X′^{L} based on the maximal level coarsening ratio a of the query current signal Y and a continuous wavelet transform CWT of a resampled raw current signal X′, which corresponds to the raw current signal X; and
calculate the feature signals Y^{L} based on the maximal level coarsening ratio a of the query current signal Y and a continuous wavelet transform CWT of the query current signal Y,
wherein the direct sequence dynamic time warping distance DSDTW is applied to the resampled raw circuit signal X′ and the query current signal Y.
